(19) United States
(12) Patent Application Publication
(10) Pub. No.: US 2021/0041409 A1
(43) Pub. Date: Feb. 11, 2021

(54) OXYGEN SENSOR ELEMENT

(71) Applicants: KOA CORPORATION, INA-SHI, NAGANO (JP); NAGAOKA UNIVERSITY OF TECHNOLOGY, NAGAOKA-SHI, NIIGATA (JP)

(72) Inventors: Tomoichiro OKAMOTO, NAGAOKA-SHI (JP); Kenichi IGUCHI, INA-SHI (JP); Ken TAKAHASHI, MINAMI-MINOWA (JP); Tetsuro TANAKA, MINOWA-MACHI (JP); Chika ITO, INA-SHI (JP)

(21) Appl. No.: 16/966,203

(22) PCT Filed: Jan. 30, 2019
(86) PCT No.: PCT/JP2019/003263

§ 371 Date: Jul. 30, 2020

(30) Foreign Application Priority Data
Jan. 31, 2018 (JP) 2018-015923

(51) Int. Cl. G01N 33/00; G01N 27/409; G01N 27/407; C04B 35/45

(52) U.S. Cl. CPC G01N 33/0036; C04B 35/4504; G01N 27/4073; G01N 27/409

(57) ABSTRACT

An oxygen sensor element made of a ceramic sintered body detects oxygen concentration based on an electric current value measured when a voltage is applied. The ceramic sintered body has a composition formula LnBa₂₋ₓSrₓCu₃O_{7-δ} generated by substituting any element selected from group 2 elements in the periodic table, such as strontium (Sr), for a part of a composition formula LnBa₂Cu₃O_{7-δ} (Ln denotes rare earth element and δ is 0 to 1). Sr substitution quantity x should satisfy an inequality constraint 0<x≤1.5. This allows provision of an oxygen sensor element that improves durability etc. without losing sensor characteristics.

### TECHNICAL FIELD

The present invention relates to a material composition of a gas (oxygen) sensor element using a ceramic sintered body.

### BACKGROUND ART

There is a demand for oxygen concentration detection in various gases, such as detection of oxygen concentration in exhaust gas of internal-combustion engines, detection of oxygen concentration for boiler combustion control, etc., and an oxygen sensor made from various materials is known as an oxygen concentration detecting element. An oxygen sensor using composite ceramics generated by mixing LnBa₂Cu₃O_{7-δ} and Ln₂BaCuO₅, for example, (Ln denotes rare earth element), which are material compositions for the oxygen sensor using a ceramic sintered body, is known (Patent Document 1).

The oxygen sensor using a wire material of the ceramic sintered body as described above is a hot spot-type oxygen sensor utilizing a hot spot phenomenon that a part of the wire material is red-heated when a voltage is applied. Such an oxygen sensor may be small, light, and may have a low cost and reduced power consumption, and future practical applications are desired.

### PRIOR ART DOCUMENTS

### Patent Documents

Patent Document 1: JP 2007-85816A (Japanese Patent No. 4714867)

### DISCLOSURE OF THE INVENTION

### Problem to be Solved by the Invention

The conventional oxygen sensor described above has a problem of durability since the wire material is easily fused as a result of hot spots generated when driving the sensor. Such fusion of the wire material may be thought of as resulting from generation of a liquid phase in local parts (particularly grain boundaries) within the hot spots.

Moreover, the characteristics of the material configuring the conventional oxygen sensor element that it easily hydrates and carbonates cause a problem that the sensor element is deteriorated due to peripheral gas components, such as water vapor or carbon dioxide gas during detection of oxygen concentration of the gas, and that durability will not be sufficient. Therefore, the conventional material composition does not allow practical application of a sensor element with improved durability.

In light of these problems, the present invention aims to provide an oxygen sensor element having high heat resistance and moisture resistance, and improved durability and reliability without losing sensor characteristics.

### Means of Solving the Problem

The present invention aims to resolve the above problems, and includes the following structure, for example, as a means for achieving the above aim. That is, the present invention is an oxygen sensor element characterized in that it is made of a ceramic sintered body and that it detects oxygen concentration based on an electric current value measured when a voltage is applied. The ceramic sintered body has a composition generated by substituting any element selected from group 2 elements in the periodic table for a part of a composition formula LnBa₂Cu₃O_{7-δ} (Ln denotes rare earth element and δ is 0 to 1).

For example, it is characterized by selecting strontium (Sr) from the group 2 elements in the periodic table. It is characterized in that when the composition generated by substituting the strontium (Sr) is represented as a composition formula LnBa₂₋ₓSrₓCu₃O_{7-δ}, for example, substitution quantity x should satisfy an inequality constraint 0<x≤1.5. It is also characterized in that a part of the composition represented as the composition formula LnBa₂₋ₓSrₓCu₃O_{7-δ}, for example, is further substituted with calcium (Ca) and lanthanum (La). It is further characterized in that, for example, a composition represented as a composition formula Ln₂BaCuO₅ (Ln denotes rare earth element) is mixed together with the composition represented as the composition formula LnBa₂₋ₓSrₓCu₃O_{7-δ}. Yet even further, for example, it is characterized in that the composition represented as the composition formula LnBa₂₋ₓSrₓCu₃O_{7-δ} has a complex perovskite structure. It is also characterized, for example, in that the ceramic sintered body is a linear body sensor element.

Furthermore, an oxygen sensor is characterized by having any one of the oxygen sensor elements described above as an oxygen concentration detecting element. For example, it is characterized in that the oxygen sensor element is stored within a protecting tube having air holes on either end.

### Results of the Invention

According to the present invention, an oxygen sensor element having high heat resistance, moisture resistance, and favorable sensor characteristics for oxygen concentration measurement, and an oxygen sensor using the element may be provided.

### BRIEF DESCRIPTION OF DRAWINGS

FIG. 1 shows exterior photos illustrating moisture resistance test results of an oxygen sensor element having the composition GdBa₂Cu₃O_{7-δ} according to a conventional example, wherein FIG. 1A illustrates the external appearance before testing, and FIG. 1B illustrates the external appearance after testing;

FIG. 2 shows exterior photos illustrating moisture resistance test results of an oxygen sensor element according to an embodiment of the present invention, wherein FIG. 2A illustrates the external appearance before testing, and FIG. 2B illustrates the external appearance after testing;

FIG. 3 is a graph giving XRD measurement results of a test sample having a conventional composition (conventional example) and a test sample (working example) according to the embodiment;

FIG. 4 is a SEM photograph illustrating SEM observation results of the broken surface of the oxygen sensor element of the conventional example after subjected to a heat-resistance test;

FIG. 5 is a SEM photograph illustrating SEM observation results of the broken surface of the oxygen sensor element according to the embodiment after subjected to a heat-resistance test;

FIG. 6 is a graph showing compared results of differential thermal analysis (DTA) measurements of the test sample having the conventional composition and test sample of the working example;

FIG. 7 is a two-component phase diagram of BaO—CuO;

FIG. 8 is a two-component phase diagram of SrO—CuO;

FIG. 9 is a diagram giving XRD measurement results of specimens, each having a different substitution quantity x of Sr (Strontium) in a composition GdBa₂₋ₓSrₓCu₃O_{7-δ};

FIG. 10 is a graph giving evaluation results of oxygen reactivity of the test sample having the conventional composition and the test sample of the working example when they are regarded as oxygen sensors;

FIG. 11 is a flowchart illustrating in a time series a manufacturing process of the oxygen sensor element according to the embodiment and an oxygen sensor using the oxygen sensor element; and

FIG. 12 is an external perspective diagram of the oxygen sensor using the oxygen sensor element according to the embodiment.

### DESCRIPTION OF EMBODIMENTS

An embodiment according to the present invention is described in detail below with reference to accompanying drawings. The oxygen sensor element according to the embodiment is comprised of a ceramic sintered body, where the sintered body is connected to a power source, thereby electric current flowing through the sintered body, and resulting in the central portion of the sintered body generating heat. Heat-generating place (called hot spot) thereof functions as an oxygen concentration detector. Moreover, the oxygen sensor having the oxygen sensor element according to the embodiment as a sensor element detects oxygen concentration based on the electric current value of current flowing through the sintered body or sensor element.

The oxygen sensor element according to the embodiment as the oxygen concentration detector has a composition generated by substituting any one element selected from group 2 elements in the periodic table, namely beryllium (Be), magnesium (Mg), calcium (Ca), strontium (Sr), barium (Ba), and radium (Ra), for a part of the composition material LnBa₂Cu₃O_{7-δ} (may be referred to as conventional composition hereafter).

In the above composition, Ln denotes rare earth element (e.g., Sc (scandium), Y (yttrium), La (lanthanum), Nd (neodymium), Sm (samarium), Eu (europium), Gd (gadolinium), Dy (dysprosium), Ho (holmium), Er (erbium), Tm (thulium), Yb (ytterbium), Lu (lutetium), etc.), and δ represents oxygen defect (0-1).

In the following explanation, a ceramic sintered body is exemplified as the oxygen sensor element according to the embodiment, wherein the ceramic sintered body is made up of a composition material GdBa₂₋ₓSrₓCu₃O_{7-δ} (substitution quantity x is 0<x≤1.5) generated by assigning Gd (gadolinium) as Ln of the conventional composition LnBa₂Cu₃O_{7-δ} and substituting Sr (strontium) for a part of the resulting composition GdBa₂Cu₃O_{7-δ}.

Results of comparatively inspecting samples manufactured using the oxygen sensor element material according to the embodiment and samples made of the conventional sensor element material are explained first. Here, green compact made from the composition described later is sintered so as to manufacture disk-shaped oxygen sensor elements (also referred to as test samples hereafter) having a diameter of approximately 16 mm and thickness of approximately 2 mm, and a moisture resistance test and a heat treatment test etc. are carried out. These samples are masses (bulk bodies) of the composition materials themselves, and are made into a form and a size that allow easy observation of change etc. in external appearance before and after the tests.

<Moisture Resistance Test Results>

Table 1 gives moisture resistance test results of the oxygen sensor element having the conventional composition and the oxygen sensor element according to the embodiment. ‘Working Example’ in Table 1 is an oxygen sensor element generated by substituting Sr (strontium) for a part of the conventional composition and assigning Gd (gadolinium) as Ln, resulting in the composition GdBa₂₋ₓCu₃O_{7-δ} (0<x≤1.5) where x=1. ‘Conventional Example’ in Table 1 is an oxygen sensor element generated by assigning Gd (gadolinium) as Ln of the conventional composition LnBa₂Cu₃O_{7-δ} without substituting Sr (strontium) for a part of the composition, namely it is an oxygen sensor element where x=0.

TABLE 1

| | XRD | | SEM |
|---|---|---|---|
| Measurement method | 40° C. 93% | 40° C. 93% | 40° C. 93% |
| Test conditions | RH 50 hours | RH 500 hours | RH 50 hours |
| Conventional example | x | x | x |
| Working example | ∘ | ∘ | ∘ |

In Table 1, x indicates that the element has degraded, and o indicates that the element has hardly degraded at all.

That is to say, in the test of leaving an element in an environment of 40° C. and 93% RH for 50 hours, the oxygen sensor element of the conventional example has degraded, while the oxygen sensor element of the working example has shown hardly any degradation. Moreover, the oxygen sensor element of the working example shows hardly any degradation even in the case of leaving the element in an environment of 40° C. and 93% RH for 500 hours.

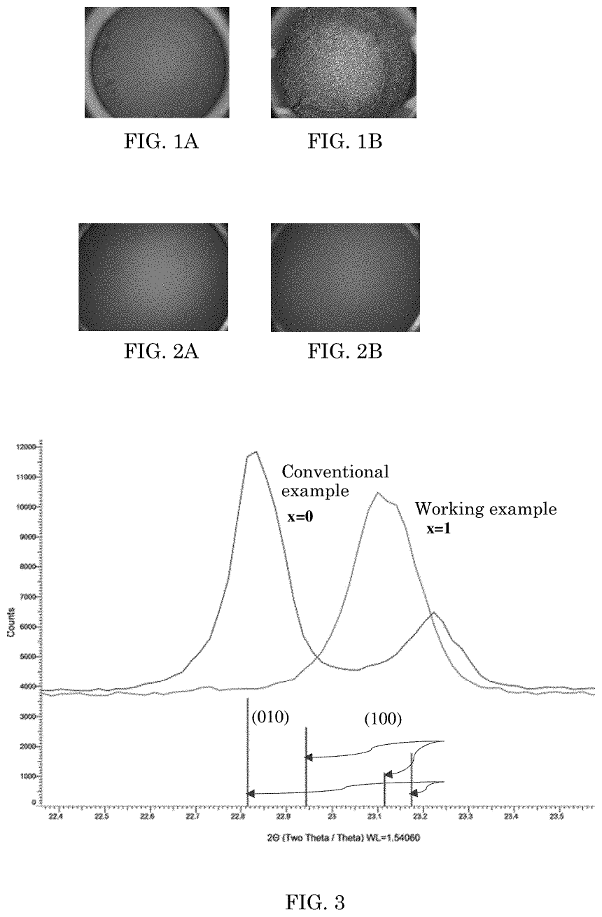

FIG. 1 shows exterior photos illustrating moisture resistance test results of the oxygen sensor element having the composition GdBa₂Cu₃O_{7-δ} according to the conventional example. FIG. 1A illustrates the external appearance of the oxygen sensor element before testing, and FIG. 1B illustrates the external appearance thereof after leaving it in an environment of 40° C. and 93% RH for 50 hours.

On the other hand, FIG. 2 shows exterior photos illustrating moisture resistance test results of the oxygen sensor element according to the embodiment, where substitution quantity x of Sr (strontium) in the composition GdBa₂₋ₓSrₓCu₃O_{7-δ} (0<x≤1.5) is 1. FIG. 2A illustrates the external appearance of the oxygen sensor element before testing, and FIG. 2B illustrates the external appearance of the oxygen sensor element after leaving it in an environment of 40° C. and 93% RH for 500 hours.

It is understood from the result of external observation that FIG. 1B shows a phenomenon that barium carbonate etc. is generated on the surface of the oxygen sensor element having the conventional composition after the moisture resistance test and that the color turns white occurs. It is clear that such phenomenon causes the oxygen sensor element to no longer react to oxygen, resulting in degradation of the element. Therefore, the oxygen sensor having the conventional composition has poor moisture resistance etc.

In contrast, as illustrated in FIG. 2B, the phenomenon that the color turns white even after the moisture resistance test is not confirmed with the oxygen sensor element according to the embodiment that is made up from a composition generated by substituting Sr (strontium) for a part of the conventional composition. This shows that the oxygen sensor element according to the embodiment has excellent moisture resistance, etc.

Measurement results of x-ray diffusion (XRD) of the oxygen sensor element according to the embodiment that is carried out to consider a mechanism improving the moisture resistance of the element will be explained. FIG. 3 is a graph giving XRD measurement results of a test sample (conventional example) of the oxygen sensor element having the conventional composition and a test sample (working example) of the oxygen sensor element according to the embodiment. Note that the vicinity of 2θ=23° is enlarged in FIG. 3.

The working example of FIG. 3 gives the XRD measurement results of the sample generated by substituting Sr (strontium) for a part of the conventional composition and assigning Gd (gadolinium) as Ln, resulting in the composition GdBa₂₋ₓSrₓCu₃O_{7-δ} (0<x≤1.5) where x=1. The working example shows, as in FIG. 3, that the peak at an orthorhombic (010) surface is decreased and that peak at a tetragonal (100) surface is increased due to the Sr substitution.

The composition material LnBa₂Cu₃O_{7-δ} of the oxygen sensor element will phase-change from orthorhombic (a≠b≠c) to tetragonal (a=b≠c) when oxygen deficiency within the crystal structure is increased. FIG. 3 illustrates diffraction patterns in the orthorhombic state and the tetragonal state, respectively. Since a≠b holds true in the orthorhombic state, both (100) and (010) surfaces exist at the same time. In the orthorhombic state, it is presumed that defects are easily generated inside of the crystals and that gaps between gratings are large. Moreover, FIG. 3 illustrates that the tetragonal diffraction pattern of the LnBa₂Cu₃O_{7-δ} complex perovskite structure is confirmed from the XRD measurements at room temperature.

<Heat-Resistance Test Results>

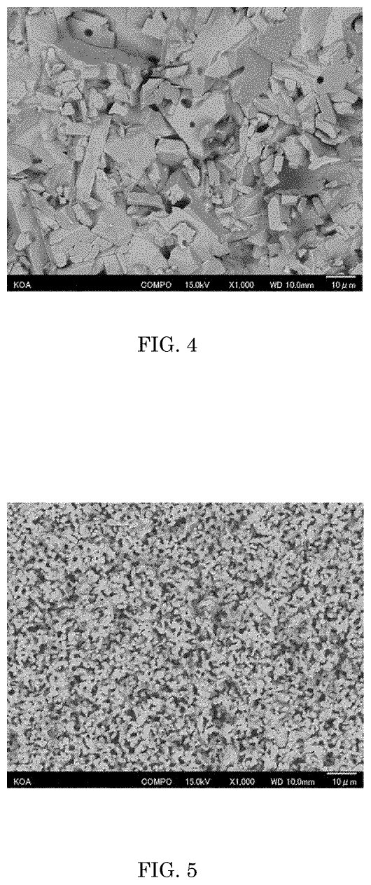

FIG. 4 is a SEM photograph illustrating SEM observation results of the broken surface of the oxygen sensor element (x=0), which is generated by assigning Gd (gadolinium) as Ln of the conventional composition LnBa₂Cu₃O_{7-δ} and then being exposed at 950° C. for 10 hours (baked at 950° C.). Moreover, FIG. 5 is a SEM photograph illustrating SEM observation results of the broken surface of a test sample of the oxygen sensor element according to the embodiment having the composition GdBa₂₋ₓSrₓCu₃O_{7-δ} (0<x≤1.5) where x=1, wherein the composition is generated by assigning Gd (gadolinium) as Ln of the conventional composition and substituting Sr (strontium) for a part of the conventional composition and then being exposed at 950° C. for 10 hours (baked at 950° C.). Note that both FIG. 4 and FIG. 5 are backscattered electron images at 1000 magnification.

As can be understood from FIG. 4 and FIG. 5, there is great difference in sintered body tissue between the test sample of the conventional composition and the test sample of the oxygen sensor element according to the embodiment even at the same heat treatment temperature. Namely, it is understood that while remarkable grain growth occurs in the oxygen sensor element of the conventional composition, grain growth is drastically suppressed in the oxygen sensor element according to the embodiment having the composition generated by Sr substitution.

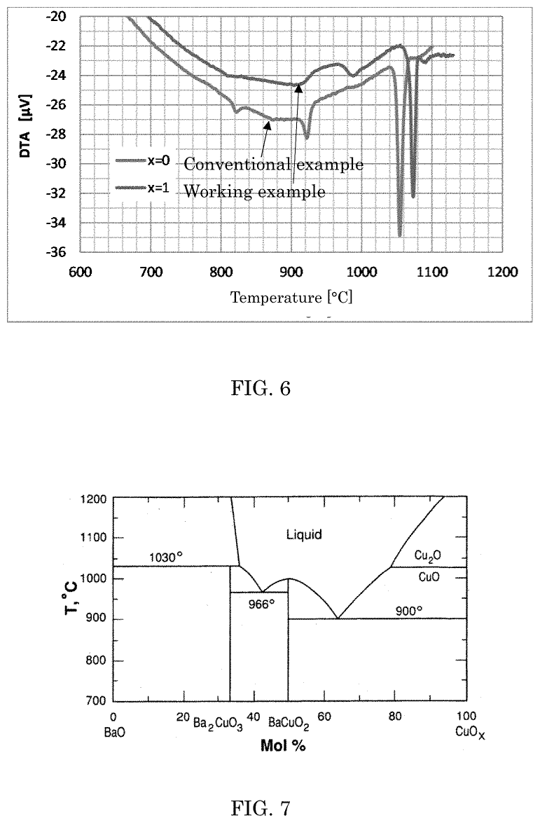

In the conventional composition (x=0), since the temperature at the hot spots of the oxygen sensor element is approximately 950° C., the sintered body structure (composition) varies during sensor operation, and thus sensor characteristics may also vary. In order to examine this mechanism, differential thermal analysis (DTA) measurement of the test sample of the conventional composition and the test sample according to the embodiment is carried out. DTA measurement results are compared in FIG. 6.

As shown in FIG. 6, it is understood from the DTA measurement that an endothermic peak in the vicinity of 920° C., which has been seen with the test sample (x=0) of the conventional composition, decreases with the test sample (x=1) according to the embodiment.

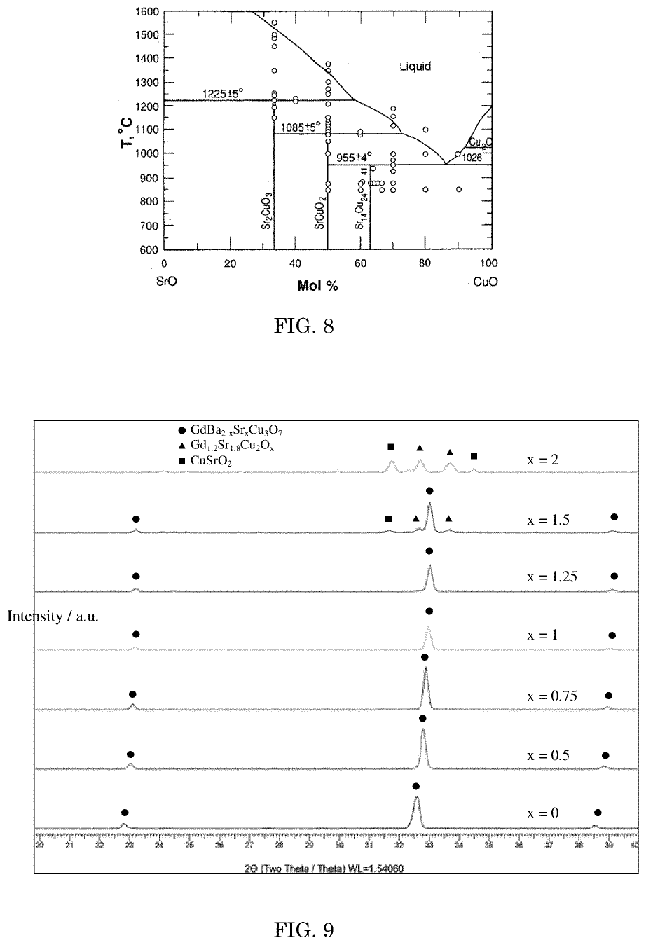

From a two-component phase diagram of FIG. 7, the endothermic peak in the vicinity of 920° C. is considered to be a liquid phase of BaO—CuO. While the eutectic point in the BaO—CuO phase diagram is at 900° C., it can be understood from a two-component phase diagram of FIG. 8 that the eutectic point in the SrO—CuO phase diagram is at 955° C., which is high. Therefore, generation of the liquid phase deriving from BaO—CuO may be reduced by substituting Strontium (Sr) for Barium (Ba) in the composition, for example. This shows that the oxygen sensor element according to the embodiment has excellent heat resistance.

<Sr (Strontium) Substitution Quantity>

Specimens having the composition GdBa₂₋ₓSrₓCu₃O_{7-δ}, which is generated by substituting Sr (strontium) for a part of the conventional composition and assigning Gd (gadolinium) as Ln (rare earth element), are manufactured, wherein substitution quantity x is set to x=0, x=0.5, x=0.75, x=1, x=1.25, x=1.5, and x=2, and XRD measurement is carried out for each specimen.

FIG. 9 gives the XRD measurement results for the specimens having the composition GdBa₂₋ₓSrₓCu₃O_{7-δ} described above where x is set to 0, 0.5, 0.75, 1. 1.25, 1.5, and 2. It is understood that a favorable range of substitution quantity x for forming the target phase of GdBa₂₋ₓSrₓCu₃O_{7-δ} should satisfy an inequality constraint 0<x≤1.5, as indicated by a ⋅ symbol in FIG. 9.

<Sensor Characteristic Evaluation Results>

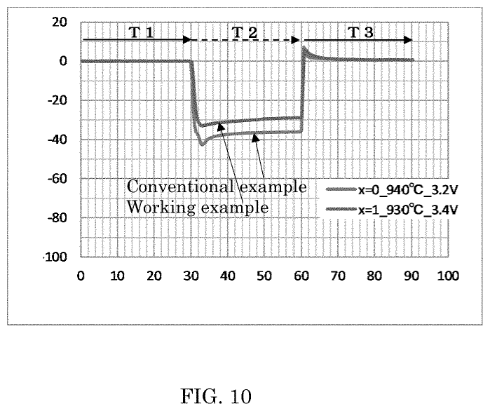

FIG. 10 gives oxygen reactivity evaluation results of the test sample (x=0) having the conventional composition and the test sample (x=1) of the working example, which function as oxygen sensors. Here, the test samples are kept in an environment of standard air (21% oxygen concentration) in time period T1 of FIG. 10. In subsequent time period T2, they are kept in an environment having 1% oxygen concentration. In subsequent time period T3, they are kept in the environment of standard air (21% oxygen concentration).

As shown in FIG. 10, the amount of change (responsiveness) in sensor output from the test sample (x=0) having the conventional composition is 36%, while 30% amount of change (responsiveness) in sensor output even from the test sample (x=1) of the working example having the composition generated by substituting Sr (Strontium) is obtained. Moreover, from the fact that the rise and fall of electric current change at respective change-points of oxygen concentration T1→T2→T3 is steep, it is understood that there is no difference in oxygen reactivity between the test sample of the conventional composition and the test sample of the working example.

This clearly shows that the same sensor characteristics (sensor output, response speed) as those of the test sample having the conventional composition can be obtained even with the sample of the working example generated by substituting Sr (Strontium) for a part of the conventional composition.

Inspection of a composition generated by substituting calcium (Ca) and lanthanum (La) for a part of the composition of the oxygen sensor element according to the embodiment that is represented by the composition formula GdBa₂₋ₓSrₓCu₃O_{7-δ} described above is carried out. As a result, it is determined that moisture resistance of such composition generated by substituting Ca and La may be improved so as to secure sensor characteristics.

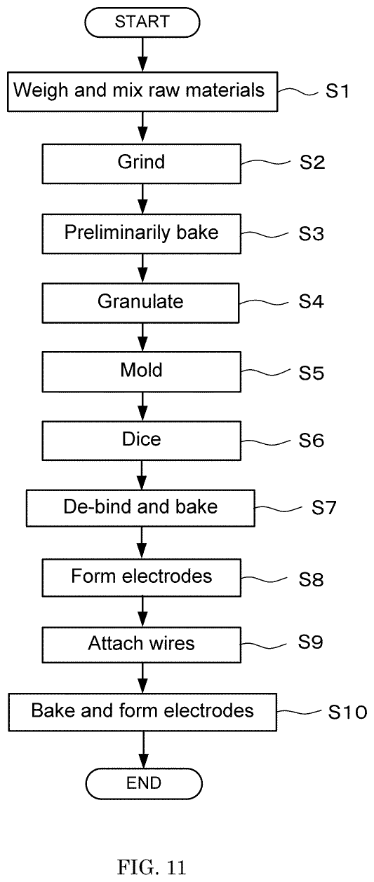

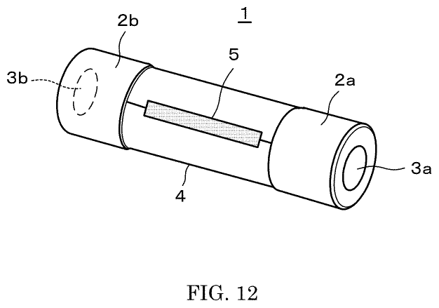

A manufacturing process for the oxygen sensor element according to the embodiment and the oxygen sensor using the element is described next. FIG. 11 is a flowchart illustrating in a time series the manufacturing process of the oxygen sensor element according to the embodiment and the oxygen sensor using the oxygen sensor element.

In Step S1 of FIG. 11, raw materials for the oxygen sensor element are weighed and mixed together. In this case, Gd₂O₃, BaCO₃, SrCO₃, and CuO, for example, are weighed using an electronic analytical scale and mixed together as materials for the oxygen sensor element so as to make a predetermined composition.

Note that Gd (Gadolinium) is exemplified in this case as Ln (rare earth element) of the oxygen sensor element material. However, another single rare earth element may be used as Ln, or otherwise multiple rare earth elements may be mixed together, namely any one of the rare earth elements may be used. Moreover, Ln₂BaCuO₅ may be further added to the mixture.

In Step S2, the raw materials of the oxygen sensor element weighed and mixed together in Step S1 are ground using a ball mill Grinding may also be carried out using a solid phase method or a liquid phase method, such as with a bead mill using beads as grinding media.

In subsequent Step S3, the ground material (raw material powder) described above is heat processed (preliminary baking) at 900° C. for 5 hours in atmospheric air. Preliminary baking is a process for adjusting reactivity and grain size. Temperature for the preliminary baking may be 880 to 970° C., and is more preferably 900 to 935° C.

Processing then progresses to a granulation step. More specifically, granulated powder is made in Step S4, wherein an aqueous solution or the like of a binder resin (e.g., polyvinyl alcohol (PVA)) is added to the preliminarily baked mixture so as to make a granulated powder.

In subsequent Step S5, a pressing pressure is applied to the granulated powder using a uniaxial press method, for example, and molded, so as to manufacture a plate member (press-molded body) having a thickness of 300 μm, for example. Molding may be carried out by a hydrostatic pressing method, hot pressing method, doctor blade method, printing method, or thin film method.

Dicing is carried out in Step S6. Dicing entails cutting the molded plate member into a predetermined product size and shape (e.g., 0.3×0.3×7 mm linear shape). The smaller the size of the oxygen sensor element, the more excellent in electric power saving, and thus the product size may be different from the size mentioned above.

In Step S7, de-binding the oxygen sensor element that has been diced in such a manner as described above is performed, and the resulting oxygen sensor element is baked in atmospheric air at, for example, 920° C. for 10 hours. Note that while the firing temperature may be 900 to 1000° C., the firing temperature may be changed according to composition since optimum temperature varies according to composition. An annealing step may be carried out hereafter.

In Step S8, both ends of the resulting oxygen sensor element are dipped and coated in sliver (Ag), and dried at 150° C. for 10 minutes, thereby forming electrodes. In Step S9, a silver (Ag) wire having a diameter of 0.1 mm, for example, is attached through a joining method such as wire bonding to the electrodes formed in Step S8 and then dried at 150° C. for 10 minutes. The terminal electrodes formed in this manner are then baked at 670° C. for 20 minutes, for example, in Step S10.

Material of the electrodes and the wire described above may be of a material other than silver (Ag), such as gold (Au), platinum (Pt), nickel (Ni), tin (Sn), copper (Cu), resin electrode, etc. Moreover, dipping the electrodes may also use a printing method or a film adhering method such as sputtering. Furthermore, electrical characteristics of the oxygen sensor element manufactured through the steps described above may also be evaluated using a four-terminal method, for example, as a final step in FIG. 11.

<Oxygen Sensor>

The oxygen sensor using the oxygen sensor element according to the embodiment has heat-generating place (hot spots) in the central portion of the oxygen sensor element, which will be oxygen concentration detectors. For example, an oxygen sensor 1 shown in FIG. 12 has a structure that an oxygen sensor element 5 is stored inside a cylindrical glass tube 4 made of heat-resistant glass, which functions as a protecting member for the oxygen sensor element. In order for the oxygen sensor 1 to be electrically connected to the outside, metal conductive caps (mouthpieces) 2a and 2b made of copper (Cu), for example, are embedded in either side of the glass tube 4.

Silver (Ag) wires attached to either end of the oxygen sensor element 5 are electrically connected to the respective conductive caps 2a and 2b using a lead-free solder and arranged such that the longitudinal direction of the oxygen sensor element 5 is the same as the axial direction of the glass tube 4 so the oxygen sensor element 5 does not touch the glass tube 4. Moreover, gas (oxygen) to be measured flows smoothly into the glass tube 4 via air holes 3a and 3b, which are provided on end surface sides of the conductive caps 2a and 3b, respectively, resulting in the oxygen sensor element 5 exposed to that gas, thereby allowing accurate measurement of oxygen concentration in the ambient atmosphere.

The outer dimensions (size) of the oxygen sensor 1 include, for example, a glass tube diameter of 5.2 mm, glass tube length of 20 mm, and air hole diameter of 2.5 mm, thereby making the oxygen sensor element having the dimensions given above (0.3×0.3×7 mm) exchangeable via the air holes of the glass tube.

Note that the protecting member of the oxygen sensor element 5 may be a ceramic case, a resin case, or the like aside from the glass tube described above. Moreover, the connection between the silver (Ag) wires attached to the oxygen sensor element 5 and the respective conductive caps 2a and 2b may be carried out through lead soldering, welding, caulking, etc.

Furthermore, while omitted from the drawing, the oxygen sensor, which uses the oxygen sensor element according to the embodiment, has a configuration for measuring oxygen concentration in the atmosphere to be measured based on the electric current measured with an ammeter since a current flows through the oxygen sensor element according to peripheral oxygen concentration when a predetermined voltage is applied to the oxygen sensor by a power source.

As described above, the oxygen sensor element according to the embodiment has a composition represented as the composition formula LnBa₂₋ₓSrₓCu₃O_{7-δ} (Ln denotes rare earth element and substitution quantity x is 0<x≤1.5), which is generated by substituting any one element selected from group 2 elements in the periodic table, such as strontium (Sr), for a part of the conventional composition represented as the composition formula LnBa₂Cu₃O_{7-δ}.

Use of such a composition raises the liquid phase melting point of SrO—CuO higher than that of the liquid phase of BaO—CuO, making it difficult for the liquid phase to generate when driving the oxygen sensor. This allows provision of an oxygen sensor element that improves heat resistance and moisture resistance of the oxygen sensor element and has high durability and reliability without losing sensor characteristics.

In addition, while an example of substituting Sr (strontium) for a part of the conventional composition is given in the embodiment described above, it may be assumed that even substitution with any one element selected from group 2 elements in the periodic table, such as beryllium (Be), magnesium (Mg), calcium (Ca), barium (Ba), and radium (Ra), gives the same results as in the case of Sr substitution.

### DESCRIPTION OF REFERENCE NUMERALS

1: Oxygen sensor
2a, 2b: Conductive cap
3a, 3b: Air hole
4: Glass tube
5: Oxygen sensor element

1. An oxygen sensor element that is made of a ceramic sintered body and that detects oxygen concentration based on an electric current value measured when a voltage is applied, wherein
the ceramic sintered body has a composition generated by substituting any element selected from group 2 elements in the periodic table for a part of a composition formula LnBa₂Cu₃O_{7-δ} (Ln denotes rare earth element and δ is 0 to 1).

2. The oxygen sensor element according to claim 1, wherein strontium (Sr) is selected from the group 2 elements in the periodic table.

3. The oxygen sensor element according to claim 2, wherein when the composition generated by substituting the strontium (Sr) is represented as a composition formula LnBa₂₋ₓSrₓCu₃O_{7-δ}, substitution quantity x should satisfy an inequality constraint 0<x≤1.5.

4. The oxygen sensor element according to claim 3, wherein a part of the composition represented as the composition formula LnBa₂₋ₓSrₓCu₃O_{7-δ} is further substituted with calcium (Ca) and lanthanum (La).

5. The oxygen sensor element according to claim 3, wherein a composition represented as a composition formula Ln₂BaCuO₅ (Ln denotes rare earth element) is mixed together with the composition represented as the composition formula LnBa₂₋ₓSrₓCu₃O_{7-δ}.

6. The oxygen sensor element according to claim 4, wherein a composition represented as a composition formula Ln₂BaCuO₅ (Ln denotes rare earth element) is mixed together with the composition represented as the composition formula LnBa₂₋ₓSrₓCu₃O_{7-δ}.

7. The oxygen sensor element according to claim 1, wherein the ceramic sintered body is a sensor element having a linear shape.

8. An oxygen sensor having an oxygen sensor element as an oxygen concentration detecting element, wherein the oxygen sensor element is made of a ceramic sintered body and that detects oxygen concentration based on an electric current value measured when a voltage is applied, wherein the ceramic sintered body has a composition generated by substituting any element selected from group 2 elements in the periodic table for a part of a composition formula LnBa₂Cu₃O_{7-δ} (Ln denotes rare earth element and δ is 0 to 1).

9. The oxygen sensor according to claim 8, wherein the oxygen sensor element is stored within a protecting tube having air holes on either end.

10. The oxygen sensor according to claim 8, wherein strontium (Sr) is selected from the group 2 elements in the periodic table.

11. The oxygen sensor according to claim 10, wherein when the composition generated by substituting the strontium (Sr) is represented as a composition formula LnBa₂₋ₓSrₓCu₃O_{7-δ}, substitution quantity x should satisfy an inequality constraint 0<x≤1.5.

12. The oxygen sensor according to claim 11, wherein a part of the composition represented as the composition formula LnBa₂₋ₓSrₓCu₃O_{7-δ} is further substituted with calcium (Ca) and lanthanum (La).

13. The oxygen sensor according to claim 11, wherein a composition represented as a composition formula Ln₂BaCuO₅ (Ln denotes rare earth element) is mixed together with the composition represented as the composition formula LnBa₂₋ₓSrₓCu₃O_{7-δ}.

14. The oxygen sensor according to claim 12, wherein a composition represented as a composition formula Ln₂BaCuO₅ (Ln denotes rare earth element) is mixed together with the composition represented as the composition formula LnBa₂₋ₓSrₓCu₃O_{7-δ}.

15. The oxygen sensor according to claim 8, wherein the ceramic sintered body is a sensor element having a linear shape.

16. The oxygen sensor element according to claim 3, wherein the composition represented as the composition formula LnBa₂₋ₓSrₓCu₃O_{7-δ} has a complex perovskite structure.

17. The oxygen sensor element according to claim 4, wherein the composition represented as the composition formula LnBa₂₋ₓSrₓCu₃O_{7-δ} has a complex perovskite structure.
